(19) United States
(12) Patent Application Publication
(10) Pub. No.: US 2007/0129269 A1
(43) Pub. Date: Jun. 7, 2007

(54) Storage system

(76) Inventors: Sandra Arce, Mine Hill, NJ (US); Silvio DiMarchi, Leonia, NJ (US); Brenda Williams-Halsey, Lake Hopatcong, NJ (US); Sheng-Fu Lee, Ramsey, NJ (US); Robert McKay, Wanaque, NJ (US); Lawrence P. Scanzillo, Upper Saddle River, NJ (US)

Correspondence Address: Johnson & Johnson, 201 TABOR ROAD, MORRIS PLAINS, NJ, 07950, US

(21) Appl. No.: 11/293,653

(22) Filed: Dec. 2, 2005

(51) Int. Cl. A61K 8/00

(52) U.S. Cl. 510/116

(57) ABSTRACT

A storage system is disclosed having (a) a case with three storage compartments provided in a tri-fold configuration; (b) at least one package of denture cleaning tablets provided in one of the three storage compartments; and (c) at least one additional denture cleaning resource provided in one of the three storage compartments.

### FIELD

The present invention relates to a storage system. The present invention more specifically relates to a storage system such as a portable storage and cleaning kit for dentures that provide users with convenient, portable, stowable and/or self-contained systems for storing denture cleaning resources.

### BACKGROUND

Denture cleaning generally is carried out either by soaking dentures in an aqueous cleansing solution and/or brushing dentures with a brush. Aqueous denture cleanser solutions are known and generally include tablets, granules, or powders that are dissolved in water to form a cleansing bath or cleansing system in water.

Denture cleansing compositions, such as effervescent tablets and powders, are known. Traditionally, these compositions have contained a variety of sulfate salts, such as bisulfates, monopersulfates, and sulfates as detergents, oxidizers and the like, and have also utilized alkali metal and alkaline earth metal halides as bleaches. Such compositions have also included perborate, carbonate and phosphate salts in varying amounts to provide effervescence and activation. Representative examples of cleansing compositions covering these various materials are set forth in U.S. Pat. Nos. 3,337,466, 3,704,227, 4,362,639 and 4,857,224. Commercial examples of denture cleaning tablets include Efferdent® denture cleanser tablets commercially available from Pfizer Consumer Healthcare of Morris Plains, N.J.

Denture cleaning tablets are generally provided to a user in boxes or containers of relatively large quantities (such as 40, 60, 120 or more tablets). Typically, 4-8 tablets are provided in an individual package or sheet. As a result, typical packaging will provide a user with 5, 10, 20 or more individual packages or sheets of denture cleaning tablets. If a user desires to use the denture cleaning tablets away from a home setting (such as while traveling), the user may bring the entire box of cleaning tablets with them. This option affords the user with containment, storage and protection of the cleaning tablets, but will likely not be practical because the user may not want to bring along unnecessary tablets and/or lose space which the box would occupy. The user could alternatively bring only one or a few individual packages with them. However, this option does not provide the user with containment, storage and protection of the cleaning tablets. The tablets may be damaged, crushed, etc., or the package may be ripped or otherwise compromised. Furthermore, a user may also require other denture cleaning resources (such as brushes for cleaning the dentures, or package openers for assisting the user to access or open the packages). Current configurations are not well suited to provide for portability, storage and/or protection of the denture cleaning tablets while also providing convenient access to and storage for other denture cleaning resources.

It would be advantageous to provide a storage system affording a portable solution for storing and protecting denture cleaning tablets against damage and accidental opening. It would further be advantageous to provide a storage system allowing convenient access to other denture cleaning resources (brushes, package openers, etc.) as needed. It would further be advantageous to provide a storage system that would assist in keeping the denture cleaning resources organized and protected from dirt and debris. It would further be advantageous to provide a storage system that provides a single location for all denture cleaning resources. It would be desirable to provide a system that provides any one or more of these or other advantageous features.

### SUMMARY

The present invention relates to storage system having (a) a case with three storage compartments provided in a tri-fold configuration; (b) at least one package of denture cleaning tablets provided in one of the three storage compartments; and (c) at least one additional denture cleaning resource provided in one of the three storage compartments.

The present invention further relates to a denture cleaning kit including (a) a case comprising first, second and third compartments; (b) a denture cleaning brush provided in the third compartment; and (c) a package opening device provided in the third compartment. The first and second compartments are configured to each receive at least one package containing a denture cleaning tablet. The first compartment and second compartment are hinged to each other along a first interface, and the first compartment and third compartment are hinged to each other along a second interface.

The present invention further relates to a kit including (a) a first compartment comprising a first member for retaining a first article in the first compartment; (b) a second compartment hingedly coupled to the first compartment and comprising a second member for retaining a second article in the second compartment; (c) a third compartment hingedly coupled to the first compartment and comprising a third member for retaining a third article in the third compartment. A locking mechanism is provided to secure the second compartment and the third compartment. At least one package of denture cleaning tablets is provided in the first compartment and partially retained by the first member, and the first compartment is the central compartment in a tri-fold configuration.

### BRIEF DESCRIPTION OF THE DRAWINGS

FIG. 1 is a front perspective view of a storage system according to an exemplary embodiment.

FIG. 2 is an exploded perspective view of the system shown in FIG. 1.

FIG. 3 is a top plan view of the system shown in FIG. 1 in an opened state.

FIG. 4 is a cross-sectional view of the system shown in FIG. 1, taken along the line 4-4.

### DETAILED DESCRIPTION

Referring to the FIGURES, exemplary embodiments of a storage system are shown. According to one exemplary embodiment shown in the FIGURES and described below, the storage system is used as a portable denture cleaning system. The portable denture cleaning system provides a user with a self-contained, portable kit to store denture cleaning resources. The system provides portability, relatively small size, and convenience to a user. Resources for denture cleaning are provided in a single container and may be easily transported. The system is especially well adapted as a travel kit to provide the user with necessary resources (such as cleaning tablets, brushes, and other accessories) for denture cleaning. The system may also be used in-home or away from home, at an office, etc.

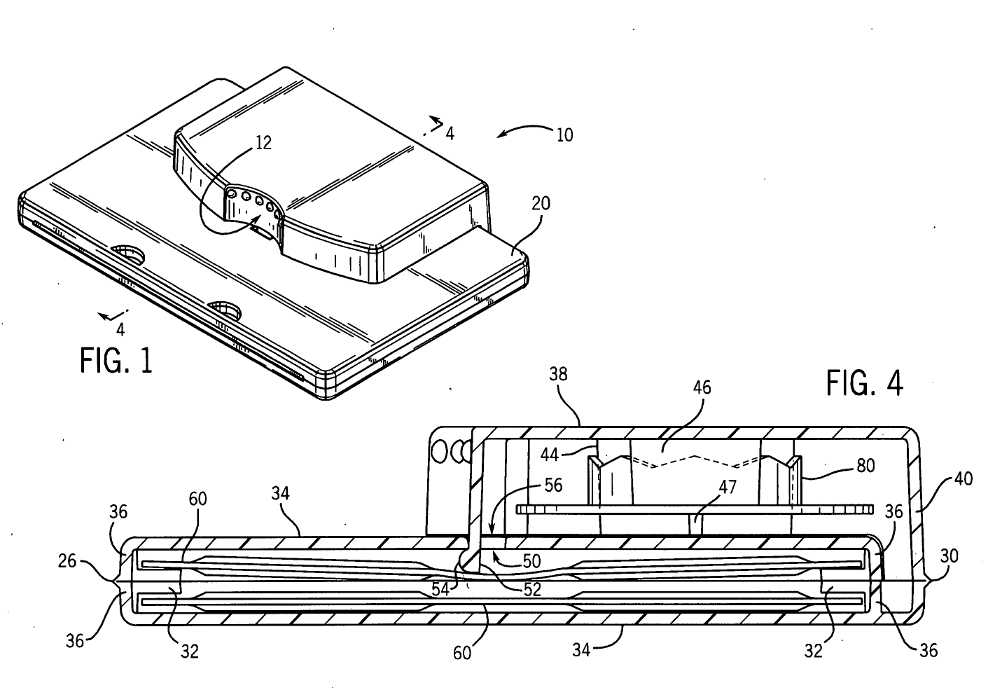

As shown in FIGS. 1-4, a storage system 10 is shown for application as a portable denture cleaning kit or for providing storage of denture cleaning resources. System 10 includes a case 20 for storage of denture cleaning resources (such as packages of denture cleaning tablets, a brush, a package opener or cutter, etc.).

Referring to FIG. 1, case 20 provides enclosure, protection and/or storage of the denture cleaning resources. Case 20 may be rigid, semi-rigid, hard sided, soft sided, resistant to deformation, or pliable. Case 20 is shown in a closed or storage position.

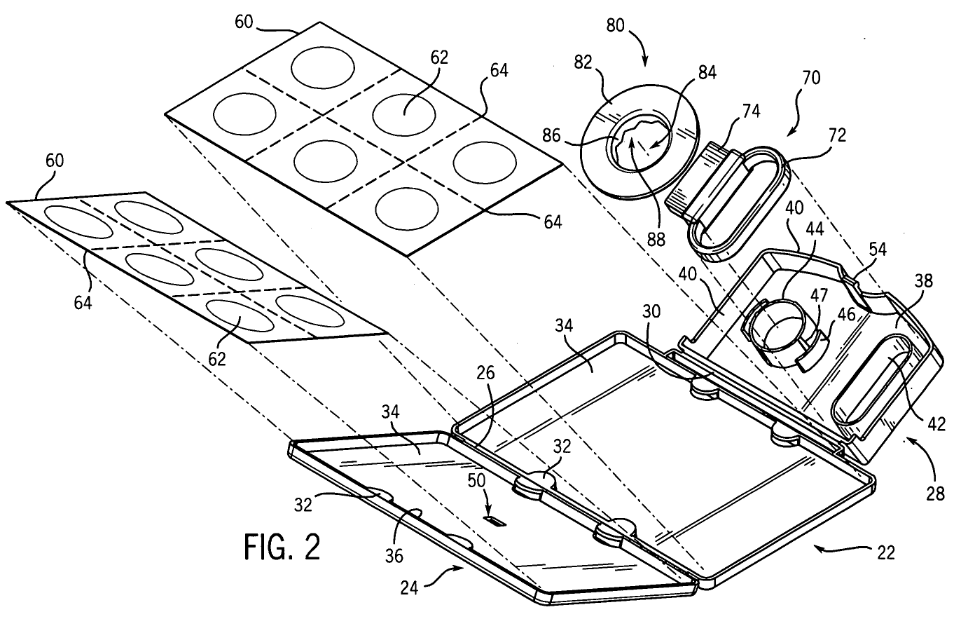

Referring to FIG. 2, case 20 is shown in an open or use position. Case 26 comprises a first compartment 22 (e.g., section, panel, member, compartment or containment area), a second compartment 24 movably coupled to first compartment 22 along interface 26, and a third compartment 28 movably coupled to first compartment 22 along interface 30. Generally, each compartment has a bottom, side walls and an open top through which a user may access the contents of each compartment. According to a particularly preferred embodiment, first compartment 22 and second compartment 24 are generally flat, planar-like containment spaces each sized to receive a package 60 of denture cleaning tablets 62. According to an exemplary embodiment, interfaces 26 and 30 are hinges (such as living hinges that are thin sections of material coupling the compartments and allowing the compartments to be opened, closed, moved, rotated, etc.). Tabs 32 (or retainers, fingers, members, etc.) are provided for first compartment 22 and second compartment 24 along the edges or the periphery of the individual compartment. According to a particularly preferred embodiment, four tabs 32 are provided in opposing pairs for each compartment. Alternatively, any number, arrangement, placement or configuration of retainers may be provided to suitable retain package 60.

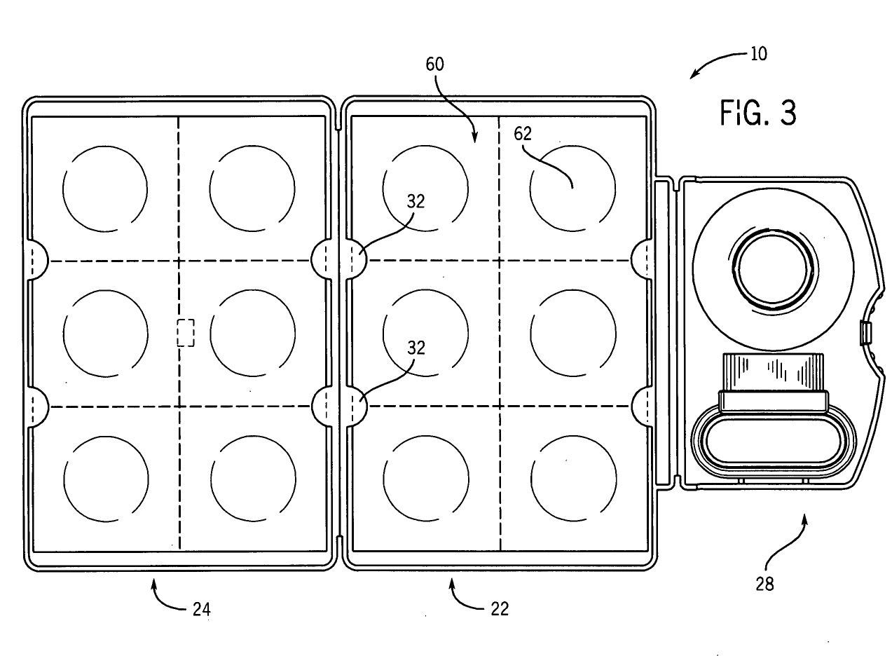

Package 60 is retained in compartments 22 and 24 by tabs 32, a base or bottom member or wall 34 of the compartment, and edges or side walls 36 of the compartment. As shown in FIG. 3, tabs 32 overlap the edges of package 60 such that package 60 may be selectively removed and/or replaced by a user, but package 60 is generally retained in position and will not fall out of compartments 22 and 24 if, for example, compartments 22 and 24 are flipped over or inverted.

Case 20 is sized to receive one or more packages of denture cleaning tablets. According to an exemplary embodiment, compartments 22 and 24 have a length of about 5¾ inches (146 mm), a width of about 3¾ inches (95 mm), and a depth of about 3/16 inch (5 mm) (which is suitable to receive a package of six denture cleaning tablets having dimensions of about 5½ inches (140 mm) in length, about 3⅝ inches (92 mm) in width, and about ⅛ inches (3 mm) in depth). The sizing of compartments 22 and 24 allows for insertion of package 60 by bending or deforming package 60 along a central perforation and allowing the periphery of package 60 to fit under tabs 32. Once under tables 32, package 60 returns back to its original shape and is retained. Compartments 22 and 24 are sized to provide sufficient clearance to allow the insertion of, and yet still securely retain, package 60. According to alternative embodiments, compartments 22 and 24 may be constructed in a variety of sizes and/or clearances to provide the desired operative effects (such as allowing for easier insertion, more secure fit, fitting larger or smaller packages, etc.).

Referring back to FIG. 2, third compartment 28 is coupled to first compartment 22 along interface 30. According to a particularly preferred embodiment, third compartment 28 and second compartment 24 are provided on opposed sides of first compartment 22. Third compartment 28 includes a base or bottom member or wall 38, and edges or side walls 40. Posts or projections 42 and 44 are coupled to wall 38. Post 42 is sized to receive and retain a handle 72 of brush 70 (such as with a friction fit, snap-fit, etc). Post 44 is sized to receive and retain package opener 80. Package opener 80 may further be retained by arms 46 (or retention tabs). Package opener 80 is received between post 44 and arms 46 to provide a secure engagement (such as a friction fit, snap-fit, etc). Alternatively, one or more ribs 47 may be provided on post 44. Ribs 47 may have a vertical flare or taper such that frictional engagement with package opener 80 increases as package opener 80 is pressed onto post 44 toward a storage position. According to an exemplary embodiment, compartment 28 has a length of about 3¾ inches (95 mm), a width of about 2 to 2½ inches (50-63 mm) and a depth of about ½ inch (13 mm).

Case 20 is secured in a locked or closed position (as shown in FIG. 1) by locking or securing mechanism 50 (such as a latch). As shown in FIG. 4, locking mechanism 50 comprises a member 52 (such as an extension or post) extending from side wall 40 of third compartment 28, a protrusion 54 provided on the end member 52, and an aperture 56 provided in bottom member 34 of second compartment 24. In a closed position, member 52 extends into aperture 56. Protrusion 54 coacts with or engages bottom member 34 near an edge of aperture 56 to prevent member 52 from pulling out of aperture 56, thereby keeping case 20 in a closed or locked position. To open case 20, a user will apply a force perpendicular to side wall 40 near member 52 (shown in FIG. 1 as finger cut-out portion 12). The force will flex or otherwise move member 52 and protrusion 54 out of engagement with bottom member 34. Case 20 is then free to move to an open position as shown in FIG. 2. According to an alternative embodiment, other locking mechanisms may be used to keep the case in a selectively open, closed or locked position such as hook and loop fasteners (such as Velcro® fasteners), snap closures, hooks, locks, etc.

As shown in FIGS. 1 and 2, case 20 folds or moves between a closed position (shown in FIG. 1) and an open position (shown in FIG. 2). The individual compartments fold relative to each other. In the closed position, first compartment 22 and second compartment 24 are in an abutting or facing relationship such that the open tops of each compartment (through which a user may access each compartment) adjoin each other, face each other, or cover each other. This arrangement results in compartments 22 and 24 closing each other off in the closed position shown in FIG. 1 (i.e., the contents of compartments 22 and 24, and the open tops, are generally enclosed or sealed off by each other in a closed position). Additionally, the open top of compartment 26 is abutting or facing bottom wall 34 of second compartment 24 in the closed position. This arrangement results in compartment 24 closing off compartment 26 (i.e., the contents of compartment 26, and the open top, is generally enclosed or sealed off by compartment 26 in a closed position).

According to an exemplary embodiment, case 20 is a single body article (i.e., unitary construction) constructed from an injection molded plastic (such as polyethylene, polypropylene, polyethylene terephthalate, etc.). According to alternative embodiments, other materials such as plastics, polymers, metals, composites, fabrics, etc. may be used. According to alternative embodiments, the case may be constructed from several separate parts attached to each other using known fasteners such as mechanical fasteners, adhesives, bonding, welding, hinges, etc.

Denture cleaning resources are stored and selectively removable from case 20. As shown in FIG. 2, denture cleaning resources include packages 60 of denture cleaning tablets 62, a denture cleaning brush 70, a package opener or cutter 80, and/or other desired resources such as swabs, wipes, strips, etc. Package 60 is a sheet-like package constructed from a foil, paper, or plastic that provides suitable boundary properties for denture cleaning tablets 62. Package 60 is shown having six individual tablets 62 provided in six individual pouches separated by score or perforation lines 64. Perforation lines 64 allow a user to tear off or separate a single tablet 62 or pouch from package 60 for use.

Package opener 80 is provided to assist a user with easily opening package 60 and gaining access to tablet 62. Package opener 80 comprises a ring-shaped body 82 having a central opening or aperture 84. Aperture 84 is sized to receive or fit a tablet 62 within aperture 84. An annular member 86 projects perpendicular to body 82. Serrations 88 (or a jagged surface, irregular surface, etc.) are provided on the end of member 86. In operation, a user will place opener 80 above tablet 62 in package 60 with the serrations 88 facing package 60. The user may then press down and/or turn opener 80 to break through, saw, or otherwise compromise package 60 and gain access to tablet 62 for use. According to an exemplary embodiment, package opener has an overall diameter of about 2 inches (51 mm) and an aperture diameter of about 1 inch (25 mm).

Brush 70 includes handle 72 and bristles 74. According to an exemplary embodiment, handle 72 has an oval shape configured to receive one or two or more fingers of a user while operating brush 70. Bristles 74 may be any of a variety of bristles (or other abrasive or textured elements) used to manually clean or brush dentures, teeth, etc. including nylon bristles, natural bristles, etc. According to an exemplary embodiment, brush 70 is a “finger-sized” brush, with handle 72 having dimensions of about 2 inches (51 mm) in width and ¾ inch (19 mm) in length. Bristles 74 are provided in three rows having dimensions of about 1¼ inches (32 mm) in width and ½ inch (13 mm) in length.

As shown in FIGS. 1-4, case 20 is provided in a tri-fold configuration (i.e., three compartments or members are configured to fold relative to each other). According to an alternative embodiment, the case may be provided in a bi-fold configuration (i.e., a first compartment configured to receive a package of denture cleaning tablets may be coupled to a second compartment configured to receive another denture cleaning resource such as a brush and/or package opener). In this configuration, the compartments are sized to cover or close each other. In other alternative embodiments, other folding arrangements may be used such as four folds, etc.

According to an alternative embodiment, a storage compartment may be provided for storage of additional resources desired by a user. Alternatively, the additional storage compartment may be sized and configured to receive a user's dentures or dental appliances (mouth-guards, retainers, etc.). The additional storage compartment may be a separately hinged panel or, alternatively, may be a designated section within one of the three compartments shown in FIGS. 1-4.

It should be appreciated that storage system 10 affords a user with a portable solution for storing and protecting denture cleaning tablets against damage and accidental opening and providing convenient access to other denture cleaning resources (brushes, package openers, etc.) as needed. System 10 keeps the denture cleaning resources organized and protected from dirt and debris, and providing a single location for all denture cleaning resources.

It is important to note that the construction and arrangement of the elements of the system as shown in the exemplary, particularly preferred and alternative embodiments is illustrative only. Although only a few embodiments of the present inventions have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or elements show as multiple parts may be integrally formed, the operation of the locking mechanism may be reversed or otherwise varied, the length or width of the elements of the system may be varied, etc. The elements and/or assemblies of the system may be constructed from any of a wide variety of materials that provide sufficient strength or durability, including any of a wide variety of moldable plastic materials in any of a wide variety of colors, textures and combinations, metals, fabrics and composites. Any numbering or naming of elements (e.g., first compartment, second compartment) is not meant to imply or limit the elements to a specified order, nor to be exclusive of other intermediate elements. Accordingly, all such modifications are intended to be included within the scope of the present inventions. Other substitutions, modifications, changes and omissions may be made in the design, operating conditions and arrangement of the preferred and other exemplary embodiments without departing from the spirit and scope of the present invention.

What is claimed is:

1. A storage system comprising:
(a) a case comprising three storage compartments provided in a tri-fold configuration;
(b) at least one package of denture cleaning tablets provided in one of the three storage compartments; and
(c) at least one additional denture cleaning resource provided in one of the three storage compartments.

2. The storage system of claim 1 wherein the at least one additional denture cleaning resource comprises at least one of a brush and a package opening device.

3. The storage system of claim 2 wherein the at least one additional denture cleaning resource further comprises a brush and a package opening device.

4. The storage system of claim 1 wherein the case is semi-rigid or rigid.

5. The storage system of claim 1 further comprising a second package of denture cleaning tablets provided in one of the three storage compartments.

6. A denture cleaning kit comprising:
(a) a case comprising first, second and third compartments;
(b) a denture cleaning brush provided in the third compartment; and
(c) a package opening device provided in the third compartment;
(i) wherein the first and second compartments are configured to each receive at least one package containing a denture cleaning tablet;
(ii) wherein the first compartment and second compartment are hinged to each other along a first interface; and
(iii) wherein the first compartment and third compartment are hinged to each other along a second interface.

7. The kit of claim 6 wherein the case is moveable between a first open position and a second closed position.

8. The kit of claim 7 wherein the first, second and third compartments each further comprise:
a bottom member; and
an opposing open area through which a user may access the compartment.

9. The kit of claim 8 wherein the case in the second closed position comprises the open area of the first compartment adjoining the open area of the second compartment.

10. The kit of claim 9 wherein the case in the second closed position further comprises the open area of the third compartment adjoining the bottom member of the second compartment.

11. The kit of claim 6 further comprising a locking mechanism provided between the second compartment and the third compartment.

12. The kit of claim 11 wherein the locking mechanism further comprises a latch.

13. The kit of claim 6 further comprising at least one package containing at least one denture cleaning tablet.

14. The kit of claim 6 wherein the case in the first compartment and the second compartment fold relative to each other and cover each other.

15. The kit of claim 6 wherein the first compartment and the third compartment fold relative to each other.

16. A kit comprising:
(a) a first compartment comprising a first member for retaining a first article in the first compartment;
(b) a second compartment hingedly coupled to the first compartment and comprising a second member for retaining a second article in the second compartment;
(c) a third compartment hingedly coupled to the first compartment and comprising a third member for retaining a third article in the third compartment;
(d) a locking mechanism provided to secure the second compartment and the third compartment;
(e) at least one package of denture cleaning tablets provided in the first compartment and partially retained by the first member;
wherein the first compartment is the central compartment in a tri-fold configuration.

17. The kit of claim 16 further comprising a brush provided in the third compartment.

18. The kit of claim 16 further comprising a package opening device provided in the third compartment.

19. The kit of claim 18 wherein the package opening device further comprises a surface configured to compromise the package of denture cleaning tablets and allow a user access to a denture cleaning tablet.

20. The kit of claim 16 wherein the first compartment, second compartment and third compartment comprise a single unitary construction.
